(19) United States
(12) Patent Application Publication
(10) Pub. No.: US 2016/0287351 A1
(43) Pub. Date: Oct. 6, 2016

(54) A MULTIFUNCTIONAL DENTAL DEVICE

(71) Applicant: 3M INNOVATIVE PROPERTIES COMPANY, St. Paul, MN (US)

(72) Inventors: Korbinian Gerlach, Gauting (DE); Ralf Kelz, Germering (DE)

(21) Appl. No.: 15/034,989

(22) PCT Filed: Nov. 10, 2014
(86) PCT No.: PCT/US2014/064820

§ 371 Date: May 6, 2016

(30) Foreign Application Priority Data
Nov. 13, 2013 (EP) 13192694.1

(51) Int. Cl. A61C 1/00; A61B 1/24; A61C 13/15; A61B 1/07

(52) U.S. Cl. CPC A61C 1/0015; A61B 1/07; A61B 1/24; A61C 19/004

(57) ABSTRACT

The invention relates to a dental device (1) comprising a housing with a tool portion (2) and a handling portion (3); at least on dental tool (49 located in the tool portion (2) of the housing; an interface device (5) for activating the at least one tool located in the handling portion of the housing, wherein the interface device (5) comprises a touch sensor with a touch surface and being adapted to translate wiping of a use's finger on the touch surface (6) into an instruction to operate the dental tool (4). The invention also relates to a method for activating a dental device.

### FIELD OF THE INVENTION

The invention relates to a multifunctional dental device comprising a housing with a handling portion and a tool portion, at least one tool on the tool portion and an interface device on the handling portion. The invention further relates to a method of operating a dental device.

BACKGROUND ART

In the dental technology, a plurality of different tools, amongst others electronic tools, are known and used for example in an dental office. One example is a light emitting tool. Such a light emitting tool is necessary since a plurality of different synthetic materials, so-called dental composites, are known which polymerize due to a metacrylate based curing mechanism when irradiated with light. For this purpose dental apparatuses are known in which light is generated, focused and transmitted to the treatment site within a patient's mouth. Furthermore in the dental technology camera systems are used for making pictures and/or movies from teeth. These different devices have to be cleaned and maintained frequently. This raises the costs in a dental office.

One example of a light emitting device is disclosed in U.S. Pat. No. 6,159,005. In this document an apparatus for photopolymerizing synthetic materials specifically dental materials, includes a light source constituted by a semiconductor base solid-state radiation emitter which emits in the blue spectral range is described. The device is formed as a small, light-weight and handy device with a built-in battery.

Furthermore, WO 2010/121723 A1 discloses a dental or medical camera comprising a plurality of components, including an optic and an image converter comprising at least one adjustable element. At least one servo adjusting device is provided for at least one of the adjustable element, in order to allow modification of camera parameter.

EP 1 887 323 A2 discloses an operating element and a method of inputting values into a medical apparatus. The medical apparatus has a touch-sensitive sensor and an operating surface. The sensor has an operating surface, which detects the presence or absence of an operating means and generates a contact value. A display of the operating element shows a current value, which is variable by means of the contact value of the sensor to a value to be set.

Although a variety of dental devices are already available there is a need for a dental device combining different functions and/or tools. Such a device should be easy to use and it should account for hygiene requirements in dentistry.

### SUMMARY OF THE INVENTION

In one aspect the invention relates to a dental device comprising:
a housing with a tool portion and a handling portion;
at least one dental tool located in the tool portion of the housing;
an interface device for activating and/or controlling the at least one tool located in the handling portion of the housing, wherein
the interface device comprises a touch sensor with a touch surface and wherein
the interface device is adapted to translate a wiping of a user's finger on the touch surface into an instruction to operate the dental tool.

In a particular aspect the invention relates to a dental device comprising:
a housing with a tool portion and a handling portion;
at least one dental tool located in the tool portion of the housing;
an interface device for activating and/or controlling the at least one tool located in the handling portion of the housing, wherein
the interface device comprises a touch sensor with a touch surface; wherein
the interface device is adapted to recognize the presence of a user's finger on the touch surface and the absence of the/any finger from the surface; wherein
the interface device is further adapted to recognize a position of the finger present on the touch surface in two dimensions relative to the touch surface; wherein
the interface device is adapted to translate a wiping of the finger on the touch surface into an instruction to operate the dental tool, and wherein
the translation of the wiping is performed based on the recognition of at least two different finger positions of the finger continuously present on the touch surface, with the instruction being in a non-correlation to absolute or relative values of individual finger positions.

The wording “non-correlation to absolute or relative values” for the purpose of the present specification means that the instruction does for example not correlate to a magnitude between two or more finger positions or the value of a particular finger position to a reference position (for example an origin or another finger position). In contrast the finger positions are preferably merely used to recognize whether a wiping is performed, and the recognition of a wiping may be used to select and/or issue an associated instruction (for example one single instruction) for operating the dental tool. This does not exclude the possibility to further recognize a dimension or direction of the wiping and dependent therefore to select and/or issue corresponding associated instructions.

The dental device according to the invention may comprise a housing with a handling portion. The handling portion is the portion that may be held in an hand of a user, e.g. a dentist or a dentist's assistant. A tool portion, which is also part of the housing, may comprise at least one tool with at least one function. For activating and controlling the dental device according to the invention it comprises an interface device with a touch sensor having a touch surface. The interface device is adapted to translate a wiping of a user's finger on the touch surface into an instruction to operate the dental tool.

In an embodiment the translation of the wiping is based on the recognition of a series of two or more different finger positions during a continuous presence of the finger on the touch surface. Preferably each of these recognized finger positions are considered in the translation of the wiping. It is noted that although for the translation of the wiping the finger positions are preferably used, the instruction derived therefrom is not correlated with such finger positions.

In an embodiment the recognition of the series of finger positions is:

(i) initiated with the recognition of a first finger position present on the touch surface,
(ii) maintained for a maximum pre-determined time period until recognition of a second finger position of the finger present on the touch surface, and
(iii) terminates upon exceeding the maximum pre-determined time period in which no further finger position is recognized of the finger present on the touch surface.

In addition the recognition of the series of finger positions is preferably terminated upon recognition of the finger becoming absent from the touch sensor. The recognition of only one finger position is preferably not interpreted by the interface device as wiping. A time period as referred to in this embodiment may for example be selected within a range of about 0.2 to 3 seconds.

Generally, the interface device may be adapted such that the translation of the wiping is based on an interpretation of a change or multiple changes of the finger position as such. This means that, for example, any individual position—for example start, intermediate or end position—of the finger alone is not as such translated into the instruction. Rather, for example, the presence of two or more different finger positions during a continuous presence of the finger on the touch surface, eventually within a pre-determined time period, may be interpreted as a change of the finger position and that change may be translated into the instruction. In addition or alternatively, the direction and/or dimension (two opposite directions) between two or more different finger positions of the finger continuously present on the touch surface, eventually within a pre-determined time period, may be translated into the instruction.

In one embodiment the interface device is adapted to recognize two simultaneous wipings, in particular caused by wiping of a first and a second finger simultaneously on the touch screen. The change of the distance between the first and second fingers present on the touch sensor may be translated into the instruction. Further, the interface device may be adapted to recognize a finger position of a first finger and simultaneous wiping of a second finger on the touch screen. The change of the distance between the first and second fingers present on the touch sensor may be translated into the instruction. The same functions may be implemented based on the recognition of three or more simultaneous wipings.

The interface device may have electronic circuitry, for example comprising a programmable microcontroller and software, for performing the translation into any instruction. The electronic circuitry further is preferably adapted to receive signals from the touch sensor related to a position in which one or more fingers are placed on the touch surface and to convert such signals into a two-dimensional finger position relative to the touch sensor.

A touch sensor or tactile sensor according to the invention is a sensor that reacts on a touch of a device or part of a human being. The touch sensor translates the touch of the device or part of the human being into a particular kind of instruction. Touch sensors including appropriate electronic circuitry and software are for example known from touch pads in computers or notebooks.

Wiping on a touch surface provides a simple way of controlling electronic devices. In addition, wiping with a finger over a touch surface does not involve much forces which enables high precision work with the dental device while wiping, which is very important especially in dental technology. For these reasons, the device according to the invention is easy and precise to use.

The instructions to operate the dental device may for example comprise at least one of activating a certain function or tool of the dental device, deactivating a certain function or tool of the dental device, controlling certain parameters like for example intensity of a light emitted or speed etc.

Integrating several interface or activating devices such as for example buttons into one interface device with a touch sensor and a touch surface may provide the additional advantage that the number of interface devices is reduced to a small number of interface devices, for example to one interface device. This saves space on the dental device itself and enables a better and more effective way of cleaning and therewith fulfillment of the hygiene requirements of the dental device, since less small edges are present on the outer surface of the device. In addition, instead of a plurality of interface devices only a small number of interface devices, such as for example only one interface device needs to be encapsulated such that adequate cleaning is possible. This does save production costs of the dental device according to the invention.

In one embodiment of the invention the touch surface is formed by a portion of the handling portion of the housing. This may provide the advantage of easy handling since this is the portion that is usually held in the user's hand and is not positioned in a patient's mouth.

In one embodiment of the invention the touch surface with the remainder of the handling portion of the housing forms a contiguous surface. Such a configuration may enable easy and effective cleaning of the dental device according to the invention, since edges or gaps on the outer surface of the device may be minimized or avoided. In addition a housing having contiguous surface may provide for a good handling since it enables the possibility of ergonomically adapting the shape of the handling portion of the housing without any extensions disturbing the shape.

In one embodiment of the invention the at least one dental tool comprises at least one light emitting device for emitting curing light and/or at least one camera. A light emitting device is an electronic device that is often used at a dentist's office for curing dental materials within the mouth of a patient. Such a device usually comprises a lot of functionalities that need to be controlled. Such functionalities are for example switching the device on and off, controlling the intensity of the light, controlling the focus of the emitted light etc. The dental device according to the invention may—either in addition or as the only tool—also provide a camera. A camera is also an often used tool in a dental office and provides a lot of functionalities that need to be controlled. Typical functions of a camera are for example switching on and off, focusing the picture, adjusting different light conditions as well as controlling certain features of the picture(s) taken. All these different tools with their functionalities may be controlled over an interface device according to the invention. It is for example possible to have one interface device that can be used to instruct all tools in the device and to control the different functionalities of the tools. This interface device can be used by wiping with a users finger over its touch surface.

In one embodiment of the invention the tool portion of the dental device according to the invention may comprise a light guide. If the dental device according to the invention comprises a light emitting device, the light needs to be guided from the device, where it is generated, into the place or site in the mouth of a patient where it is needed. A simple way of doing that is by using a light guide that is adapted to guide the light in the above described manner. On the other side the light guide needs to be adapted to fulfill the high hygiene standards of dentistry. The light guide may be rotatable with respect to the handling portion of the housing. Such a configuration may have the advantage of providing a more comfortable device since it is not necessary to rotate the entire tool in order to position the tool in an appropriate way. With such feature it is possible for the user to keep the handling portion in the hand as is and to adjust the position of the light guide relative to the handling portion.

In one embodiment of the invention the interface device comprises at least one touch and/or tactile sensor. As already mentioned above, it is possible to integrate all interface devices into one touch sensor, providing the advantage of a very lean device. It is of course also possible to have several sensors integrated into the device, such as for example one sensor for each tool. An according dental device may therefore for example provide one touch sensor for a light emitting device and a separate touch sensor for a camera.

In one embodiment of the invention the interface device may comprise an incorporated button functionality. A button functionality is a functionality that translates a certain kind of tapping (single tapping, double tapping or multiple tapping), which may be defined as a short contact of a device or a part of a human being, such as for example a finger, with the touch surface, into a corresponding pre-determined instruction to operate the dental tool. The button functionality may for example be used to switch the dental device from one mode into another mode, e.g. to switch the device on and off or to switch from a camera mode into a light emitting mode.

In one embodiment of the invention the interface device may operate as a capacitive sensor. A capacitive sensor is a sensor that reacts on capacitive changes. Those capacitive changes may for example be triggered by a user's finger wiping and/or tapping on the touch surface. Touching the touch surface of the interface device results in a distortion of the electrostatic field of the interface device, which is measurable as a change in capacitance and which may be transferred into any kind of electronic instruction.

In one embodiment of the invention the interface device comprises at least one touch pad and/or touch screen. Usually a touch pad, which may also be called track pad is a pointing device with a special surface that can translate a motion and position of a device or user's finger to, e.g. a relative position on a screen of a computer or notebook. A touch pad according to the invention may be defined as a device that can translate a motion and position of a device user's finger into an instruction to operate the dental device. A touch screen is an electronic visual screen or display that can be controlled through simple and/or multiple touch gestures by touching the screen with one or more fingers or equivalent devices. The touch screen can be used to react to what is displayed and to control how it is displayed. The touch screen enables the user to interact directly with what is displayed on the screen, rather than using a separate device, such as a mouse or a touch pad. For the invention a touch screen as an interface device may have the advantage of directly showing the status of the dental tool or tools according to the instruction that has been given to the device through the touch screen.

In one embodiment of the invention the handling portion comprises at least one indicating device to display a status of at least one tool. The indicating device may comprise a visual signal. The visual signal may provide information about the status/mode etc. of the tool or tools of the dental device. Having an indicating device on the dental device enables proper and save handling of the dental device. The indicating device may also provide a tactile signal and or a sound signal.

In one embodiment of the invention the indicating device may be incorporated into the interface device. Incorporating the indicating device into the interface device provides a compact solution with interface and indicating device being at the same location. Such a construction provides the advantage of only having the need of encapsulating one area of the housing. In another embodiment of the invention the indicating device may be separate from the interface device. Such a construction may be advantageous because the indicating device will not be covered by a user's finger when the user is interacting with the interface device.

In one embodiment the indicating device may comprise at least one light emitting diode. A light emitting diode is a very cost effective and easy means to establish an indicating device. The light emitting diode may easily be integrated into the interface device or separate from the interface device into the housing. The light emitting diode may provide one or more color(s) for indicating a different mode and/or status of tool(s) and/or functionality(ies).

In one embodiment of the invention the housing may form a hermetically sealed inner space. Such a construction enables cleaning according to the hygiene requirements of dental devices.

According to another aspect the invention provides a method of operating a dental device with the above described features, the method comprising the following steps:
a user holding the dental device in his hand;
the user positioning at least one finger on the touch surface of the interface device and
the user wiping the at least one finger on the touch surface of the interface device in order to give an instruction to operate the dental tool, preferably, with the instruction being in a non-correlation to absolute or relative values of individual finger positions.

The method according to the invention allows easy and effective and therefore accurate way of operating the dental device according to the invention comprising at least one tool with at least one functionality.

### BRIEF DESCRIPTION OF THE FIGURES

FIG. 1 is a three dimensional view of one embodiment of a dental device according to the invention and

FIG. 2 are schematic views of an interface device according to the invention.

### DETAILED DESCRIPTION OF THE INVENTION

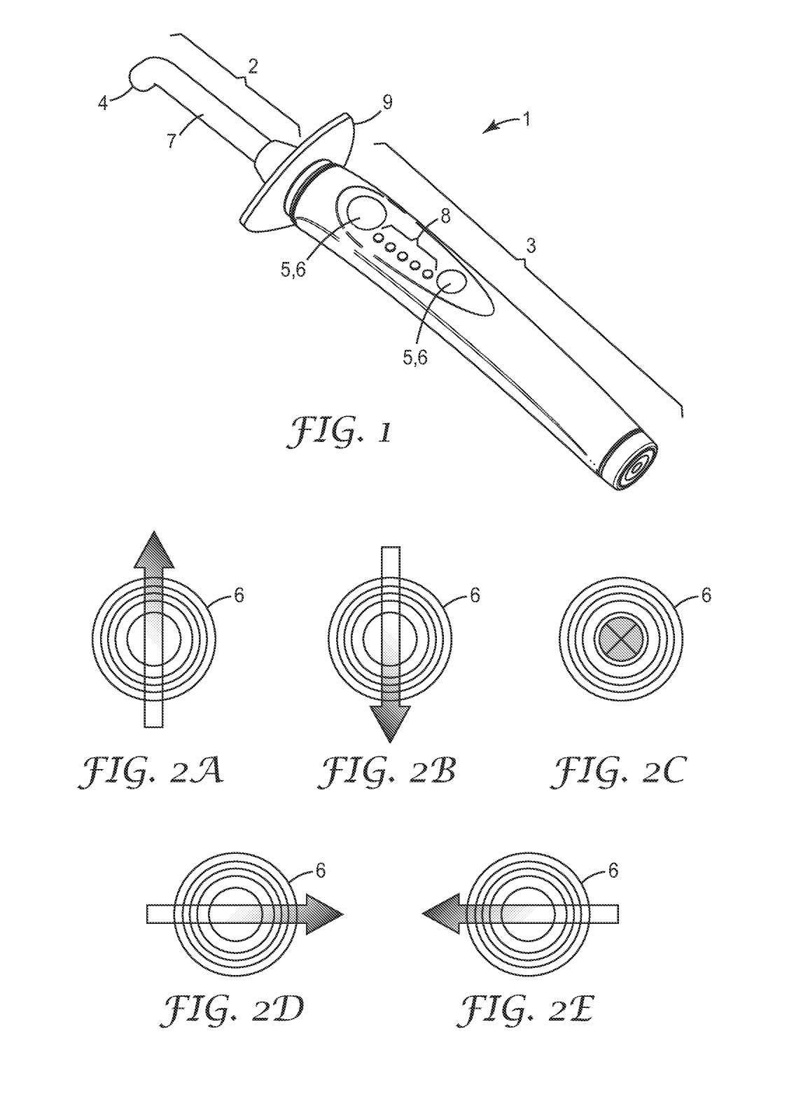

FIG. 1 is a three dimensional view of one embodiment of a dental device 1 according to the invention. The dental device 1 comprises a housing with a tool portion 2 and a handling portion 3. The handling portion 3 has an elongated shape which can easily be held in a user's hand. The handling portion 3 further comprises two interface devices 5 each providing a circular touch surface 6. Between the two touch surfaces a series of five indicating devices 8 is organized in a row, each indicating device providing information about a tool or a functionality of a tool. Tool portion 2 and handling portion 3 are separated from each other through an eye protection shield 9. The tool portion 2 itself comprises a light emitting tool with a light guide 7.

FIG. 2 are schematic views of an interface device according to the invention showing different possible wiping motions which each may be translated into a different instruction to the dental device. Possible wiping motions are from the lower end of the touch surface 6 to its upper end, parallel to the extension of the dental device (see FIG. 2a). Another possible wiping motion may be from the upper end of the touch surface 6 to its lower end, parallel to the extension of the dental device (see FIG. 2b). Another possible wiping motion is from the right edge of the touch surface 6 to its left edge, vertical to the extension of the dental device (see FIG. 2d), or the opposite way (see FIG. 2c). Finally FIG. 2 indicates the possibility of providing a touch surface with a button functionality as indicated by the cross in the touch surface (see FIG. 2c).

All the different wiping motions may be linked to one specific instruction to the operation mode of the dental device. It is of course also possible to activate the interface device with a diagonal motion and/or to use the button/functionality with simple, double and/or multiple touches (clicks) on the touch surface. Furthermore it is possible to have differently shaped touch surfaces which allow for a still bigger variety of different motions.

One example of a method of operating the above device may be the following: a user takes and holds the above described dental device 1 in his hand. When the dental device 1 has been brought into the position where it is needed, the user may position his finger on at least one of the touch surfaces 6. By wiping his finger over the touch surface 6 the user may now operate the dental device 1 in the appropriate way.

1. A dental device 1 comprising:
a housing with a tool portion and a handling portion;
at least one dental tool located in the tool portion of the housing;
an interface device for activating the at least one tool located in the handling portion of the housing, wherein
the interface device comprises a touch sensor with a touch surface wherein
the interface device is adapted to recognize the presence of a user's finger on the touch surface and the absence of the/any finger from the surface; wherein
the interface device is further adapted to recognize a position of the finger present on the touch surface in two dimensions relative to the touch surface; wherein
the interface device is adapted to translate wiping of the finger on the touch surface into an instruction to operate the dental tool, and wherein
the translation of the wiping is performed based on the recognition of at least two different finger positions of the finger continuously present on the touch surface, with the instruction being in a non-correlation to absolute or relative values of individual finger positions.

2. The device of claim 1, wherein the touch surface is formed by a portion of the handling portion of the housing.

3. The device of claim 1, wherein the touch surface with the remainder of the handling portion of the housing forms a contiguous surface.

4. The device of claim 1, wherein the at least one dental tool comprises at least one light emitting device for emitting curing light and/or at least one camera.

5. The device of claim 1, wherein the tool portion comprises a light guide.

6. The device according to claim 5, wherein the light guide is rotatable with respect to the handling portion of the housing.

7. The device of claim 1, wherein the interface device comprises at least one touch and/or tactile sensor.

8. The device of claim 1, wherein the interface device comprises an incorporated button functionality.

9. The device of claim 1, wherein the interface device operates as a capacitive sensor.

10. The device of claim 1, wherein the interface device comprises at least one touch pad and/or touch screen.

11. The device of claim 1, wherein the handling portion comprises at least one indicating device to display a status of at least one tool.

12. The device of claim 11, wherein the indicating device is incorporated into the interface device.

13. The device of claim 11, wherein the indicating device is separate from the interface device.

14. The device of claim 11, wherein the indicating device comprises at least one light emitting diode.

15. The device of claim 1, wherein the housing forms a hermetically sealed inner space.

16. A method for activating a dental device of claims 1, wherein the method comprises the following steps:
a user holding the dental device in his hand;
the user positioning at least one finger on the touch surface of the interface device and the user wiping the at least one finger on the touch surface of the interface device in order to give an instruction to operate the dental tool, with the instruction being in a non-correlation to absolute or relative values of individual finger positions.
